(19) United States
(12) Patent Application Publication
(10) Pub. No.: US 2025/0332339 A1
(43) Pub. Date: Oct. 30, 2025

(54) DEVICES, SYSTEMS, AND METHODS FOR FLOW COMPENSATION IN A FLUID MANAGEMENT SYSTEM

(71) Applicant: BOSTON SCIENTIFIC SCIMED, INC., Maple Grove, MN (US)

(72) Inventors: Cassandra Anne Hailson, Marlborough, MA (US); John O'Donnell, Tipperary (IE); Anant Vaidyanathan Subramaniam, Boston, MA (US); Damien Fitzgerald, Tipperary (IE); Teagan Jo Walter, Boston, MA (US); Troy Velazquez, Salem, NH (US); Jessica Williams, Marlborough, MA (US)

(73) Assignee: BOSTON SCIENTIFIC SCIMED, INC., Maple Grove, MN (US)

(21) Appl. No.: 19/191,259

(22) Filed: Apr. 28, 2025

Related U.S. Application Data
(60) Provisional application No. 63/640,089, filed on Apr. 29, 2024.

(51) Int. Cl. A61M 3/02; A61B 1/015

(52) U.S. Cl. CPC A61M 3/0258; A61B 1/015; A61M 3/0201; A61M 2205/3334; A61M 2205/3355; A61M 2205/3365; A61M 2205/502

(57) ABSTRACT

A fluid management system including a fluid management console and a fluid cassette configured to be received within the fluid management console. The console may comprise a housing, a controller housed within the housing, an inflow pump disposed within the housing, and a user input interface. The fluid cassette configured to be received within a receptacle of the housing of the fluid management console and to provide a flow of fluid to a medical device. The controller of the fluid management console may be configured to control the inflow pump to provide a flow of fluid at a predetermined flowrate.

### CROSS-REFERENCE TO RELATED APPLICATIONS

The application claims the benefit of U.S. Provisional Patent Application Ser. No. 63/640,089, filed on Apr. 29, 2024, the disclosure of which is incorporated herein by reference.

### TECHNICAL FIELD

The disclosure is directed to a fluid management system. More particularly, the disclosure is directed to flow compensation in a fluid management system.

### BACKGROUND

Flexible ureteroscopy (fURS), gynecology, and other endoscopic procedures require the circulation of fluid for several reasons. Surgeons today deliver the fluid in various ways such as, for example, by hanging a fluid bag and using gravity to deliver the fluid, filling a syringe and manually injecting the fluid or using a peristaltic pump to deliver fluid from a reservoir at a fixed pressure or flowrate via a fluid management system. Fluid management systems may adjust the flowrate and/or pressure at which fluid is delivered from the reservoir based on data collected from a procedural device, such as, but not limited to, an endoscope. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In a first example, a fluid management system may comprise a fluid management console and a fluid cassette configured to be received within a receptacle of the housing of the fluid management console. The fluid management console may comprise a housing, a controller housed within the housing, an inflow pump disposed within the housing, and a user input interface. The fluid cassette may be configured to provide a flow of fluid to a medical device. The controller of the fluid management console may be configured to control the inflow pump to provide a flow of fluid at a predetermined flowrate.

Alternatively or additionally to any of the examples above, in another example, if a pump pressure reaches a predetermined pump pressure, the controller may be configured to adjust a speed of a motor of the pump.

Alternatively or additionally to any of the examples above, in another example, if an intraluminal pressure reaches a predetermined intraluminal pressure, the controller may be configured to adjust a speed of a motor of the pump.

Alternatively or additionally to any of the examples above, in another example, controlling the inflow pump may comprise reducing a revolutions per minute (RPM) of a motor of the pump.

Alternatively or additionally to any of the examples above, in another example, controlling the inflow pump may comprise stopping a motor of the pump.

Alternatively or additionally to any of the examples above, in another example, controlling the inflow pump may comprise reversing a direction of a motor of the pump.

Alternatively or additionally to any of the examples above, in another example, the predetermined pump pressure may be less than a maximum pump pressure.

Alternatively or additionally to any of the examples above, in another example, the maximum pump pressure may be a setpoint pump pressure plus an offset.

Alternatively or additionally to any of the examples above, in another example, the offset may be 150 mmHg.

Alternatively or additionally to any of the examples above, in another example, the predetermined intraluminal pressure may be less than a maximum intraluminal pressure.

Alternatively or additionally to any of the examples above, in another example, the controller may be configured to maintain the predetermined flowrate when a tool may be inserted into a working channel of the medical device.

Alternatively or additionally to any of the examples above, in another example, the controller may be configured to maintain the predetermined flowrate when an outflow resistance is increased above a baseline outflow resistance.

Alternatively or additionally to any of the examples above, in another example, the controller may be configured to control the inflow pump to provide a flow of fluid at the predetermined flowrate by increasing and/or decreasing a pump pressure.

Alternatively or additionally to any of the examples above, in another example, when the pump pressure is above a setpoint pump pressure, a notification may be displayed on the user input interface.

Alternatively or additionally to any of the examples above, in another example, the pump pressure may be increased by a restriction in flow of a fluid.

In another example, a fluid management system may comprise a fluid management console and a fluid cassette. The fluid management console may comprise a housing, a controller housed within the housing, an inflow pump disposed within the housing, and a user input interface. The fluid cassette may be configured to be received within a receptacle of the housing of the fluid management console and to provide a flow of fluid to a medical device. The controller of the fluid management console may be configured to allow a pump pressure of the inflow pump to increase above a setpoint pump pressure in response to a flow restriction to provide a flow of fluid at a predetermined flowrate.

Alternatively or additionally to any of the examples above, in another example, if the pump pressure reaches a predetermined pressure greater than the setpoint pressure, the controller may be configured to adjust a speed of a motor of the pump.

Alternatively or additionally to any of the examples above, in another example, if an intraluminal pressure reaches a predetermined intraluminal pressure greater than the setpoint pressure, the controller may be configured to adjust a speed of a motor of the pump.

In another example, a fluid management system may comprise a fluid management console and a fluid cassette. The fluid management console may comprise a housing, a controller housed within the housing, an inflow pump disposed within the housing and having a pump motor, and a user input interface. The fluid cassette may be configured to be received within a receptacle of the housing of the fluid management console and to provide a flow of fluid to a medical device. When a pump pressure is below a predetermined pump pressure and an intraluminal pressure is below a predetermined intraluminal pressure, the controller of the fluid management console may be configured to operate the pump motor at a fixed revolutions per minute.

Alternatively or additionally to any of the examples above, in another example, if either the pump pressure increases above the predetermined pump pressure or the intraluminal pressure increases above the predetermined intraluminal pressure, the controller may be configured to slow, stop, or reverse the pump motor.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a perspective view of the front side of the fluid cassette of the disposable tubing set of FIG. 2;

FIG. 4 is perspective view of the rear side of the fluid cassette of the disposable fluid tubing set of FIG. 2;

FIG. 5 is a cross-sectional view of the fluid cassette of FIGS. 3 and 4 showing the internal fluid pathway therethrough;

FIG. 6 is an enlarged cross-sectional view of a portion of the fluid cassette of FIG. 5 showing the damping chamber;

FIG. 7 is a schematic view of an illustrative medical device that may be used in conjunction with the fluid management system of FIGS. 1-6;

FIG. 8 is a flow chart of an illustrative method for controlling an irrigation fluid in a flow-control mode;

FIG. 9 is an illustrative graph of the pressure of the fluid management system and the RPMs 404 of the inflow pump during a portion of an example procedure;

FIGS. 10A and 10B are graphs of the pressure of the fluid management system and the RPMs of the inflow pump during a portion of another example procedure;

FIGS. 11A and 11B are graphs of the pressure of the fluid management system and the RPMs of the inflow pump during a portion of another example procedure;

FIG. 12A is a cross-sectional view of a portion of the fluid cassette of FIGS. 3 and 4 showing the internal fluid pathway with low inflow pump pressure; and

FIG. 12B is a cross-sectional view of a portion of the fluid cassette of FIGS. 3 and 4 showing the internal fluid pathway with high inflow pump pressure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ Elite endoscope device using pressure and/or temperature data from the endoscope or other endoscopic device. Direct regulation of the intraluminal pressure during a medical procedure may allow the fluid management system to safely drive pump pressures of up to 600 mmHg to ensure no loss of flow during the procedure when tools are inserted into the working channel of the endoscope device. In some procedures, blood and/or debris may be present in the body cavity, which may negatively affect image quality through the endoscopic device. Fluid flow (e.g., irrigation) through the endoscopic device may be used to flush the body cavity to improve image quality. In some procedures, the body cavity may be relatively small and irrigation fluid may flow continuously. These fluid management systems may be pressure driven such that when a tool is introduced into the working channel of an endoscope, the flowrate of fluid through the working channel, and consequently visualization, decreases. Similarly, if outflow (e.g., from the kidney in fURS procedures) is restricted, flowrate may also reduce. In practice, the change in flowrate may require the user to continually adjust the input pressure to compensate for tools in the working channel. This may take time and efforts. Further, such adjustments may be imprecise and inconsistent across different users. The present disclosure is directed towards a fluid management system which provides flow-driven irrigation to maintain consistent visualization with and without tools.

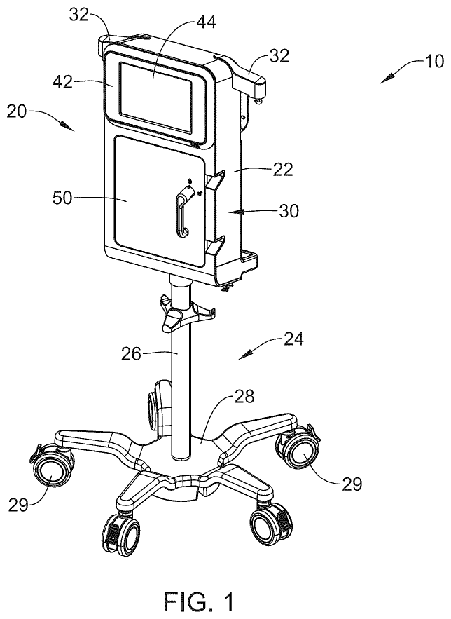

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the LithoVue™ Elite endoscope, or other endoscope. In some instances, the endoscope may include a temperature sensor to provide intraluminal temperature feedback to the fluid management system 10, a pressure sensor to provide intraluminal pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms (such as, but not limited to, a max pressure alarm), information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate or the fluid pressure based on previously set parameters, as discussed herein.

An illustrative fluid management unit may include one or more fluid container supports, such as fluid supply source hanger(s) 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

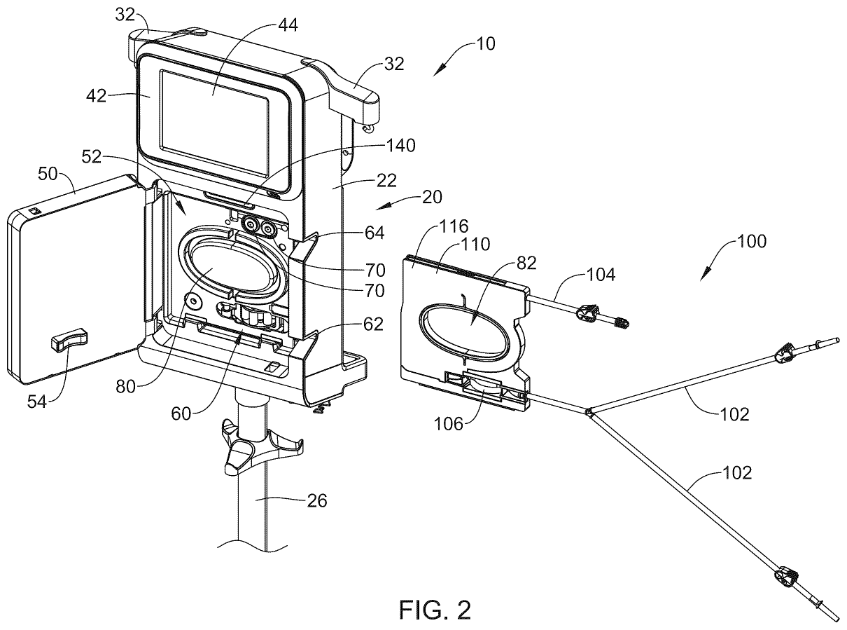

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. The inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target or predetermined fluid flowrate or target fluid pressure based on a set of system operating parameters. In some embodiments, the controller 30 may be configured to control the inflow pump 60 to maintain a desired fluid pressure at the treatment site or a predetermined flowrate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may the adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

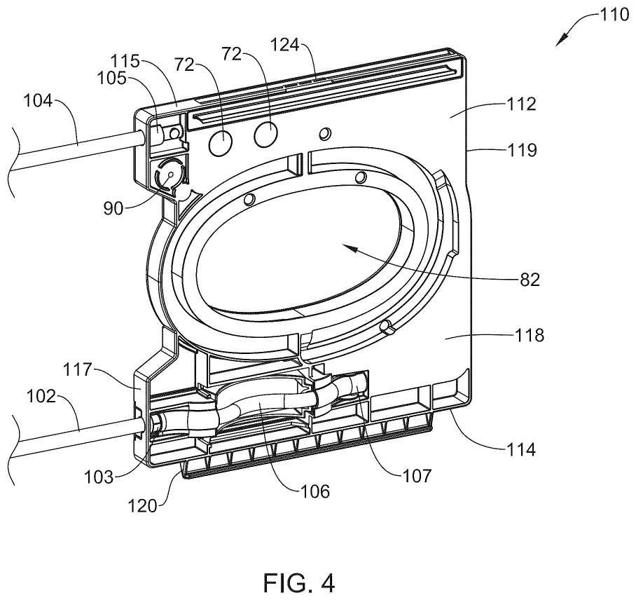

The console 20 may further include one or more additional sensors, such as a pressure sensor and/or a bubble sensor. For instance, the console 20 may include a pressure sensor 70, illustrated as a pair of pressure sensors, configured to monitor a system pressure (i.e., pump pressure) of fluid exiting the cassette 110 and flowing through the outflow tubing 104 to a surgical site. The fluid cassette 110 may include a corresponding pressure sensor interface 72, such as a flexible membrane, (shown in FIG. 4) that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44.

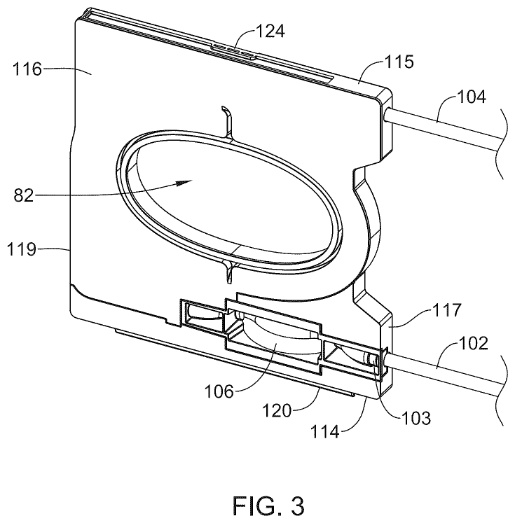

Additional features of the cassette 110 of the fluid tubing set 100 are illustrated in FIGS. 3 and 4. The fluid cassette 110 may include a housing 112 defining a fluid pathway through an interior of the housing 112. The fluid cassette 110 may include a front face 116 and a rear face 118 opposite the front face 116. The front face 116 is configured to face the door 50 of the console 20 when loaded in the receptacle 52, and the rear face 118 is configured to face a rear wall of the receptacle 52 of the console 20 when loaded in the receptacle 52. The fluid cassette 110 may also include an upper edge 115 and a lower edge 114 opposite the upper edge 115. The fluid cassette 110 may also include a first side edge 117 and a second side edge 119, opposite the first side edge 117. Both, the inflow tubing 102 and the outflow tubing 104 may extend from the first side edge 117. The housing 112 of the fluid cassette 110 may also include an opening 82, such as an oval opening, extending through the housing 112 from the front face 116 to the rear face 118. The opening 82 may extend a majority of the length of the housing 112 (i.e., a majority of the distance between the lateral edges of the housing 112) and/or a majority of the height of the housing 112 (i.e., a majority of the distance between the upper edge and the lower edge of the housing 112), in some instances. The opening 82 may be configured to receive an elevated portion of the rear wall of the receptacle 52, shown in FIG. 1 as the fluid warming system 80. The elevated portion of the rear wall of the receptacle 52 may be an oval shape sized to fit through the oval shaped opening 82 of the housing 112 of the fluid cassette 110 when the fluid cassette 110 is in its loaded position in the receptacle 52. In embodiments, in which the console 20 lacks a fluid warming system, the elevated portion of the rear wall of the receptacle 52 may still be present. Insertion of the elevated portion of the rear wall of the receptacle 52 through the opening 82 of the fluid cassette 110 may facilitate proper alignment of the fluid cassette 110 in the receptacle 52, for example.

In some embodiments, the fluid cassette 110 may include a fluid inlet port 103 and a fluid outlet port 105 located at a lateral side of the fluid cassette 110 accessible from the first side edge 117 of the fluid cassette 110. The fluid inlet port 103 may be coupled to the inflow tubing 102 and the fluid outlet port 105 may be coupled to the outflow tubing 104, with the inflow tubing 102 and the outflow tubing 104 extending laterally from the first side edge 117. The fluid inlet port 103 may be located below (e.g., closer to the lower edge 114) than the fluid outlet port 105. Thus, the inflow tubing 102 may extend laterally from the first side edge 117 at a location below (e.g., closer to the lower edge 114) than the location that the outflow tubing 104 extends from the first side edge 117. The cassette 110 may define an internal fluid pathway through an interior of the cassette housing 112 of the cassette 110 between the fluid inlet port 103 and the fluid outlet port 105. In embodiments of the fluid management system 10 including fluid warming capabilities, the internal fluid pathway may include the susceptor. The length of flexible pump tubing 106 of the cassette 110, configured to engage and be compressed by the rollers of the inflow pump 60, may extend from the fluid inlet port 103 to a connection 107 of the cassette 110 leading to the fluid pathway defined through the interior of the cassette 110. The flexible pump tubing 106 may be a discrete length of tubing separate from the inflow tubing 102 and the outflow tubing 104. In some instances, the flexible pump tubing 106 may extend through an arcuate pathway between the fluid inlet port 103 to the connection 107, such that the flexible pump tubing 106 follows the rotational path of the rollers of the inflow pump 60. The inlet port 103, the outlet port 105, and/or the connection 107 may be formed as a portion of the cassette housing 112 or may be formed separately and connected thereto.

The fluid cassette 110 may also include an air vent valve 90 configured to release air from the interior of the fluid cassette 110 to atmosphere. For example, the fluid cassette 110 may include an air vent including a hydrophobic membrane, allowing air, including bubbles entrained in the fluid, to pass through the hydrophobic membrane while preventing fluid within the fluid cassette 110 to pass therethrough. The air may then be vented to atmosphere through the air vent valve 90.

The fluid cassette 110 may also include one or more retention features configured to interact with the console 20 to retain the fluid cassette 110 in the receptacle 52 of the console 20. For example, the fluid cassette 110 may include a retention tab 120 extending from a lower edge of the housing 112 of the fluid cassette 110 and/or a retention tab 124 extending from an upper edge 115 of the housing 112 of the fluid cassette 110, configured to engage mating retention features of the console 20, as described in U.S. Provisional Application Ser. No. 63/597,481, entitled Fluid Management System With Disposable Fluid Cassette, filed on Nov. 9, 2023, the contents of which are hereby incorporated by reference in their entirety.

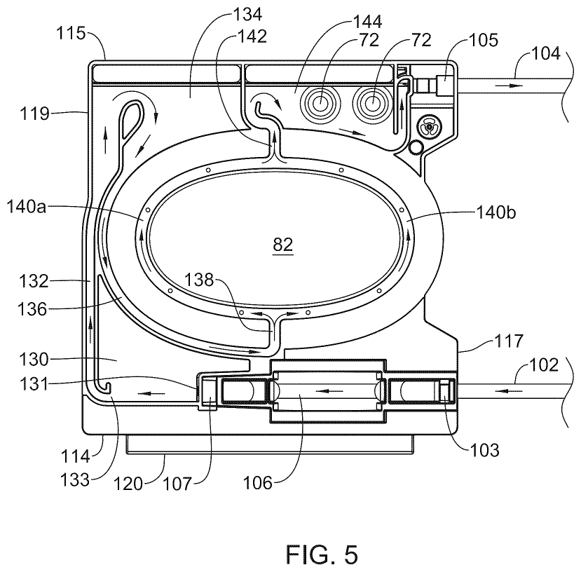

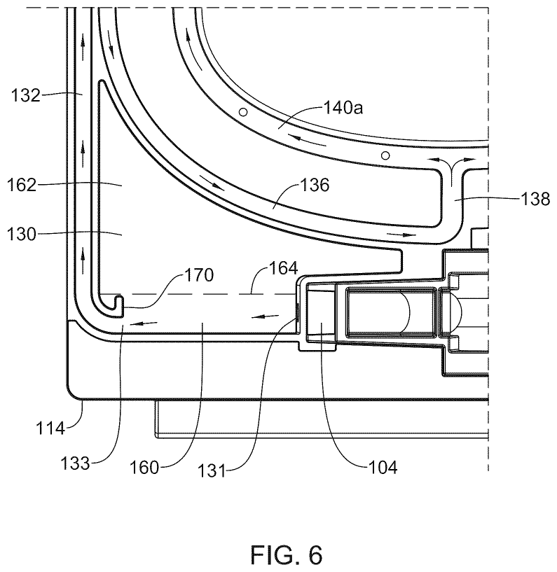

The internal flow pathway through the fluid cassette 110 is shown with arrows in the cross-sectional view of FIG. 5. Fluid flows into the interior of the fluid cassette 110 through the fluid inlet port 103 from the inflow tubing 102, and then passes through the pump tubing 106 as the pump tubing 106 is cyclically compressed by the rollers of the inflow pump 60. The fluid then flows into a fluid damping chamber 130 configured to reduce pressure fluctuations of the pulsatile fluid flow exiting the pump tubing 106 created by the inflow pump 60, and thus smoothen the fluid flow as the fluid exits the fluid damping chamber 130. The fluid damping chamber 130 may include a single fluid inlet 131 and a single fluid outlet 133. The single fluid inlet 131 and the single fluid outlet 133 may be located on opposite sides of the fluid damping chamber 130, such that fluid flows into the fluid damping chamber 130 through the fluid inlet 131 and flows out of the fluid damping chamber 130 through the fluid outlet 133. More details of the fluid damping chamber 130 will be described herein, in regard to FIG. 6.

As fluid exits the fluid damping chamber 130 through the fluid outlet 133 the fluid flows upward through the ascending fluid pathway 132. The ascending fluid pathway 132 interconnects the fluid damping chamber 130 with a first air vent chamber 134. The fluid then exits the first air vent chamber 134 in a downward direction along a descending fluid pathway 136. As shown in FIG. 5, the descending fluid pathway 136 may be an arcuate pathway extending from an upper region above the oval opening 82 to a lower region below the oval opening 82.

The fluid may then enter a bifurcated fluid pathway 140a/140b from the descending fluid pathway 136 as the fluid passes through a fluid warmer inlet channel 138 interconnecting the descending fluid pathway 136 and the bifurcated fluid pathway 140a/140b. The bifurcated fluid pathway 140a/140b includes a first fluid warming pathway 140a extending from the fluid warmer inlet channel 138 in a first direction and a second fluid warming pathway 140b extending from the fluid warmer inlet channel 138 in a second, generally opposite direction. The first fluid warming pathway 140a may extend around a first portion of the oval opening 82 on a first side of the oval opening 82 and the second fluid warming pathway 140b may extend around a second portion of the oval opening 82 on a second, opposite side of the oval opening 82. The bifurcated fluid pathway 140a/140b may then converge at a fluid mixing channel 142 located above the oval opening 82. Thus, the oval opening 82 may be located between the fluid mixing channel 142 and the fluid warmer inlet channel 138, such that the fluid mixing channel 142 is positioned above the oval opening 82 and the fluid warmer inlet channel 138 is positioned below the oval opening 82. Fluid may flow upward from the fluid mixing channel 142 into a second air vent chamber 144. The fluid may then exit the second air vent chamber 144 to the outflow tubing 104 through the outlet port 105.

FIG. 6 is an enlarged view of the portion of the fluid cassette 110 including the fluid damping chamber 130. During usage of the fluid cassette 110, a volume of fluid 160 may fill the lower portion of the fluid damping chamber 130 while a volume of air 162 is trapped in the upper portion of the fluid damping chamber 130. The fluid level 164 is the direct interface between the volume of fluid 160 and the volume of air 162. The fluid damping chamber 130 may be designed to substantially smoothen the pulsatile fluid flow from the inflow pump 60 for fluid flows up to 800 ml/min, in some instances. For example, it has been found that sizing the fluid damping chamber 130 such that the volume of air 162 is at least 38 ml substantially smoothens the pulsatile fluid prior to exiting the fluid damping chamber 130. Accordingly, the fluid damping chamber 130 may be sized to provide a volume of air 162 of 38 ml or more, or 40 ml or more, in some instances. For instance, the fluid damping chamber may be sized to provide a volume of air 162 of about 38 ml to 42 ml, during use.

Furthermore, as noted above, the fluid damping chamber 130 may include a single fluid inlet 131 and a single fluid outlet 133 located on opposite sides of the fluid damping chamber 130, such that fluid flows into the fluid damping chamber 130 through the fluid inlet 131 and flows out of the fluid damping chamber 130 through the fluid outlet 133. The fluid inlet 131 and the fluid outlet 133 may be positioned near a base of the fluid damping chamber 130. The fluid damping chamber 130 may be configured such that the upper extent of the fluid outlet 133 is lower (i.e., closer to the lower edge 114 of the fluid cassette 110) than the upper extent of the fluid inlet 131. This ensures that the fluid level 164 is above the upper extent of the fluid outlet 133 such that air from the volume of air 162 is not pulled out of the fluid damping chamber 130 into fluid exiting the fluid damping chamber 130 though the fluid outlet 133 into the ascending fluid pathway 132, which could otherwise occur at high flowrates. In some instances, the fluid outlet 133 may include a lip 170 extending upward from the upper extent of the opening of the fluid outlet 133 into the fluid damping chamber 130. The lip 170 may have any desired height. In some instances, the height of the lip 170 may be sized such that the fluid level 164 is above the upper extent of the lip 170. In other instances, the fluid level 164 may impinge the lip 170.

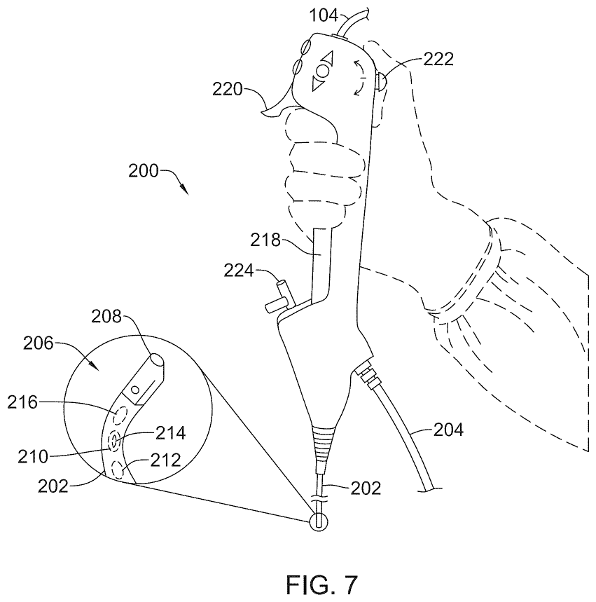

FIG. 7 illustrates aspects of the medical device 200 that may be used in conjunction with the fluid management system 10. In the illustrated embodiments, the medical device 200 may be a ureteroscope such as a LithoVue™ Elite endoscope, another intraluminal pressure sensing endoscope, or other endoscope. However, other medical devices, such as another endoscope, may be used in addition to or in place of a ureteroscope. The medical device 200 may be configured to deliver fluid from the fluid management system 10 to the treatment site via an elongate shaft 202 configured to access the treatment site within the patient. In some embodiments, the inflow pump 60 may be in fluid communication with the elongate shaft 202. The elongate shaft 202 may include one or more working lumens for receiving a flow of fluid or other medical devices therethrough. The medical device 200 is connected to the fluid management system 10 via one or more supply line(s) 104 (e.g., a tube), as shown in FIGS. 1 and 4 for example.

In some embodiments, the medical device 200 may be in electronic communication with a workstation (not explicitly shown) via a wired connection 204. The workstation may be in wired or wireless communication with the controller 30 of the fluid management system 10. In some embodiments, the workstation may be a multi-use component (e.g., used for more than one procedure) while the medical device 200 may be a single use device, although this is not required. In some embodiments, the workstation may be omitted and the medical device 200 may be electronically coupled directly to the controller 30 of the fluid management system 10.

As shown in FIG. 7, the medical device 200 may include one or more sensors proximate a distal end 206 of the elongate shaft 202. For example, the medical device 200 may include a pressure sensor 208 at a distal tip of the elongate shaft 202 to measure intraluminal pressure within the treatment site. The medical device 200 may also include other sensors such as, for example, a temperature sensor 210, a Fiber Bragg grating optical fiber 212 to detect stresses, and/or an antenna or electromagnetic sensor 214 (e.g., a position sensor). In an illustrative embodiment, the distal end 206 of the medical device 200 may also include at least one camera 216 to provide a visual feed to the user on the display screen of the workstation. In another embodiment, the medical device 200 may include two cameras 216 having different communications requirements or protocols so that different information may be relayed to the user by each camera 216. When so provided, the user may switch back and forth between cameras 216 at will through the touch screen interface 42 and/or the workstation. While not explicitly shown, the elongate shaft 202 may include one or more working lumens for receiving the fluid and/or other medical devices.

The medical device 200 includes a handle 218 coupled to a proximal end of the elongate shaft 202. The handle 218 may have a fluid flow on/off switch 220, which allows the user to control when fluid is flowing through the medical device 200 and into the treatment site. The handle 218 may further include other buttons 222 that perform other various functions. For example, in some embodiments, the handle 218 may include buttons to control the temperature of the fluid. It will be understood that while the exemplary embodiment describes a ureteroscope, the features detailed above may also be directly integrated into a cystoscope, an endoscope, a hysteroscope, or virtually any device with an image capability. In some embodiments, the medical device 200 may also include a drainage port 224 which may be connected to a drainage system. Some illustrative drainage systems are described in commonly assigned U.S. Patent Application Publication No. 2018/0361055, titled AUTOMATED FLUID MANAGEMENT SYSTEM, the disclosure of which is hereby incorporated by reference.

As the pressure sensor 208 relays the intraluminal pressure to the controller 30, the controller 30 may be configured to safely drive a constant flowrate of irrigation fluid through the scope. For example, in some cases, it may be desirable to provide a consistent or predetermined flowrate of fluid regardless of whether or not a tool is present in the working channel. The controller 30 may be configured to allow a user to enter a flow-control or flow compensation mode when intraluminal pressure data is available (e.g., from the pressure sensor 208). The intraluminal pressure data may be used to prevent intraluminal pressures from exceeding a safe limit. When the controller 30 is operating the fluid management system 10 in a flow-control or flow compensation mode, a user may observe a substantially constant flowrate or minimal disruption in the flowrate whether or not a tool is in the working channel. Generally, the controller 30 may be configured to operate the inflow pump 60 at a fixed RPM. Operating the inflow pump 60 at a fixed RPM may cause the pressure to fluctuate as tools are inserted and/or removed from the working channel. It is contemplated that the controller 30 may receive pressure measurements from within the fluid management system 10 (e.g., from pressure sensors 70) and from the pressure sensor 208 on the endoscope 200. The controller 30 may be configured to adjust the RPMs of the inflow pump 60 if either a max pressure is approached or reached.

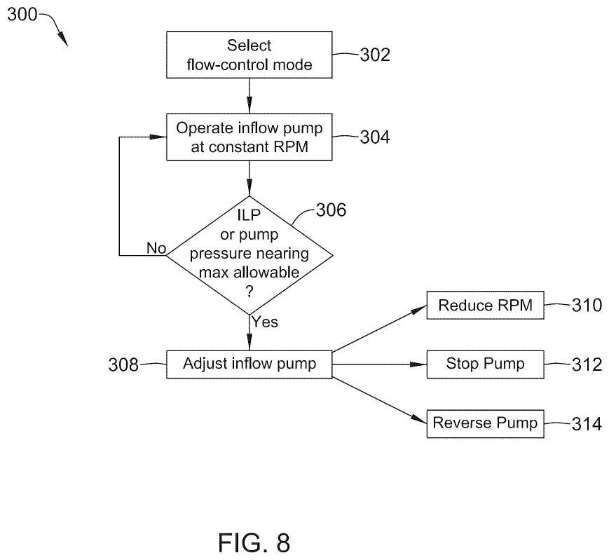

FIG. 8 is a flow chart of an illustrative method 300 for controlling the irrigation fluid in a flow-control or flow compensation mode. To begin, the flow-control mode may be selected, as shown at block 302. In some embodiments, the flow-control mode may be manually selected by a user at the touch screen interface 42. In other embodiments, the flow-control mode may be automatically initiated by the control when a particular procedure mode is selected. The controller 30 may be configured to store operating parameters for one or more flow-control modes. Operating parameters may include, but are not limited to, RPM of the inflow pump 60, maximum pressure within the fluid path of the fluid management system 10, maximum intraluminal pressure (ILP), inflow pump pressure setpoint, or the like. In some cases, one or more the operating parameters may be defined by a pre-existing program stored on or communicated to the controller 30. In other cases, one or more of the operating parameters may be input at the touch screen interface 42 by the user. After selection of the flow-control mode, the procedure may begin. As the user is operating the endoscope, the controller 30 may operate the inflow pump 60 at a constant RPM to provide a constant volumetric flowrate of the fluid, as shown at block 304. For example, the flowrate may be determined by the RPMs of the inflow pump 60.

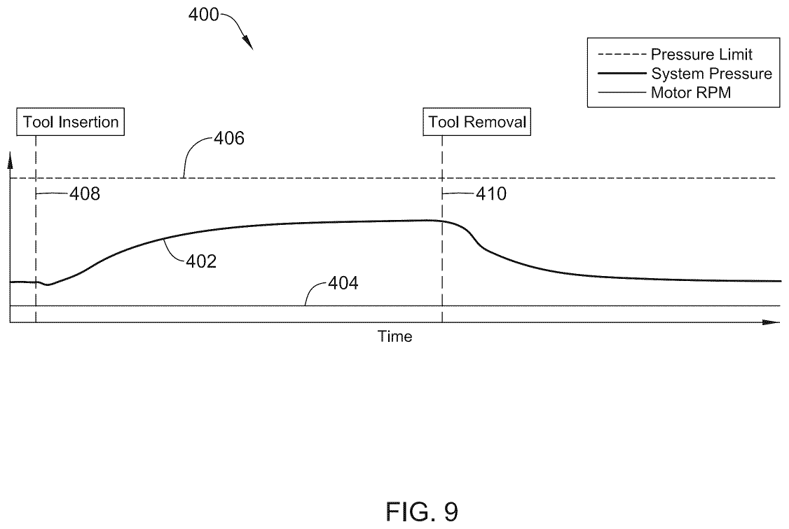

FIG. 9 is an illustrative graph 400 of the pressure 402 of the fluid management system 10 and the RPMs 404 of the inflow pump 60 during a portion of an example procedure. The fluid management system 10 may include a maximum pump pressure 406. The maximum pump pressure 406 may be a pressure value greater than the setpoint pump pressure, and in some cases the maximum pump pressure 406 may be an offset set as a percentage of the setpoint pump pressure or an absolute offset of the setpoint pump pressure (e.g., a given increase from the setpoint pump pressure). In some cases, the maximum pump pressure may be 150 mmHg above the setpoint pump pressure. For example, if the pump pressure setpoint is 150 mmHg, the controller 30 may allow the actual pump pressure to go up to 300 mmHg. This is just an example. The pump pressure setpoint may be less than or greater than 150 mmHg. Further, the allowable offset may be less than or greater than 150 mmHg, as desired. The offset may be selected to provide flow compensation (e.g., a relatively uniform flowrate whether a tool is present or not) while reducing the risk of a large bolus of fluid entering the fluid pathway when pressure is reduced (e.g., tool is removed).

At the beginning of the procedure, a tool is not present in the working channel of the endoscope 200. A tool is inserted into the working channel a first time point, as shown at 408. Once the tool has been inserted into the working channel, the pump pressure 402 begins to rise until a steady state has been achieved. The pressure may remain elevated until the tool is removed from the working channel. The tool may be removed at a second time point, after the first time point, as shown at 410. Once the tool has been removed, the pump pressure 402 begins to drop until a steady state has been achieved. The pressure may remain lowered until another tool is inserted into the working channel. As can be seen in FIG. 9, the RPMs 404 of the pump remain constant regardless of whether or not the tool has been inserted into the working channel. This may allow the fluid management system 10 to deliver a relatively constant flowrate of fluid. This may help increase visibility for the user during an entirety of the procedure.

Any increase in pump pressure above the setpoint pump pressure may be flow compensation. It is contemplated that the controller 30 may be configured to provide a visual or audio alert or notification to the user, for example, via the touch screen interface 42 whenever the flow compensation is “active”, or the actual pressure is above the setpoint pressure. In some cases, operational conditions may cause flow compensation to be active even when a tool is not inserted into the working channel. For example, an outflow scenario which generates more outflow resistance than a well-placed 11 F inner diameter/13 F outer diameter ureteral access sheath may result in flow compensation being active without a tool being present in the working channel. Some illustrative scenarios which may result in an increased outflow resistance relative to a well-placed 11 F/13 F ureteral access sheath may include longer 11 F/13 F access sheaths, 10 F/12 F access sheaths, more restrictive ureters where no access sheath is used, or the like. While the preceding scenarios are more specific to endourological procedures, similar scenarios in which outflow resistance is increased relative to a baseline scenario may occur in other endoscope procedures. As the controller 30 may compensate for increased outflow resistance, flow-restricting accessories (e.g., stopcocks or the like) may be advised against as the flow compensation of the current system may counteract an attempted reduction in fluid flow using the flow-restricting accessories.

Returning to FIG. 8, as the fluid management system 10 is operated in the flow-control mode, the controller 30 may continually monitor the pump pressure as well as the intraluminal pressure to determine if either is approaching or nearing their respective maximum pressures, as shown at block 306. If both pressures are within safe or operational limits (e.g. above the pump pressure setpoint but below the offset maximum pressure threshold (e.g., offset)), the fluid management system 10 may continue operating in the flow-control mode with no further modifications to operating parameters. If either the pump pressure approaches or exceeds the maximum operating pump pressure (e.g., pressure setpoint plus offset) or the intraluminal pressure approaches or exceeds a maximum pressure threshold (e.g., a user defined maximum or a preset maximum intraluminal pressure), the controller 30 may be configured to adjust the inflow pump 60, as shown at block 308. It is contemplated that the controller 30 may be configured to adjust the inflow pump 60 prior to either the pump pressure or the intraluminal pressure reaching their respective maximum. This may help prevent the maximum pressure from being exceeded if there is a temporal delay between adjustment of the inflow pump 60 and a reduction in pressure. For example, the controller 30 may be configured to adjust the inflow pump 60 at a predetermined pressure that is less than the maximum pressure for either the pump pressure or the intraluminal pressure.

In some examples, the controller 30 may be configured reduce the RPMs of the inflow pump 60, as shown at block 310. Reducing the RPMs of the inflow pump 60 may reduce the flowrate and thus lower the pressure. In another example, the controller 30 may be configured to completely stop the inflow pump, as shown at block 312. This may stop a flow of fluid to the endoscope 200. In yet other examples, the controller 30 may be configured to reverse the direction of the pump 60, as shown at block 314. This may move fluid in an opposite direction which may remove fluid from the fluid damping chamber 130 to lower pressure in the fluid pathway of the fluid management system 10. In some embodiments, there may be a time delay between detecting the pump pressure and/or intraluminal pressure approaching or exceeding a maximum pressure and acting to lower the pressure. A time delay may allow the controller 30 to determine if the pressure increase is due to the position of the endoscope 200 (e.g., the distal end region 206 contacting or pushing into body tissue, etc.) or an actual increase in the pressure within the fluid pathway and/or ILP. The time delay may help prevent unnecessary changes in the operation of the inflow pump 60. The time delay may be 5 seconds or less, 10 seconds or less, 15 seconds or less, 20 seconds or less, or the like. In some examples, the time delay may be greater than 20 seconds.

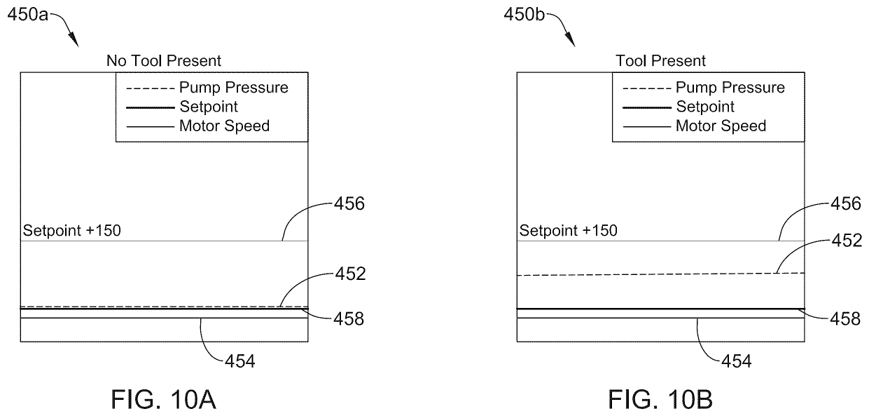

FIGS. 10A and 10B are graphs 450a, 450b of the pressure 452 of the fluid management system 10 and the RPMs 452 of the inflow pump 60 during a portion of an example procedure. In FIG. 10A, there is no tool present in the working channel while in FIG. 10B a tool has been inserted into the working channel of the endoscope. In FIG. 10A, when no tool is present, the pump pressure 452 is at or below the setpoint pressure 458 for an entirety of the portion of the procedure shown. As the pump pressure 452 does not approach the maximum pump pressure 456, the inflow pump 60 is operated at a substantially constant RPM 454 to maintain a constant flow of fluid to the working site. In FIG. 10B, when a tool is present, the pump pressure 452 increases and is maintained above the setpoint pressure 458 for an entirety of the portion of the procedure shown but remains below the maximum pump pressure 456. Thus, the inflow pump 60 is operated at a substantially constant RPM 454 even with the tool in the working channel to maintain a constant flow of fluid to the working site even with the pressure increasing above the setpoint pressure 458.

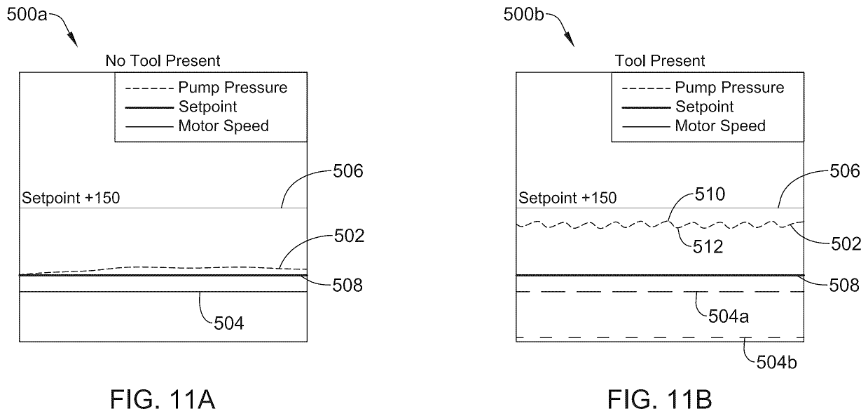

FIGS. 11A and 11B are graphs 500a, 500b of the pressure 502 of the fluid management system 10 and the RPMs 504 of the inflow pump 60 during a portion of another example procedure. In FIG. 11A, there is no tool present in the working channel while in FIG. 11B a tool has been inserted into the working channel. In FIG. 11A, when no tool is present, the pump pressure 502 is at or above the setpoint pressure 508 for an entirety of the portion of the procedure shown. However, the pump pressure 502 does not approach the maximum pump pressure 502 and the inflow pump 60 is operated at a substantially constant RPM 504 to maintain a constant flow of fluid to the working site. In FIG. 11B, when a tool is present in the working channel of the endoscope, the pump pressure 502 is above the setpoint pressure 508 for an entirety of the portion of the procedure shown. In some points during the portion of the procedure, the pump pressure 502 begins to approach the maximum pump pressure 506. In such an instance, corrective action is automatically taken at the pump motor to lower the pressure giving the pump pressure curve 506 a plurality of peaks 510 and valleys 512. For example, as the pump pressure 502 approaches the maximum pump pressure 506, the pump motor may be turned off, slowed, or reversed, as shown at 504b. Once the pump pressure 502 drops to a predetermined pressure or by a predetermined amount, the pump motor may be operated at the setpoint RPMs, as shown at 504a. It is contemplated that the drop in the pump pressure 502 may be temporally offset from the slowdown or deactivation of the pump motor. This may be due to the time it takes pressure within the system to dissipate. As can be seen, the inflow pump 60 may be oscillated between two or more motor speeds to maintain the pump pressure 502 at a safe level while also maintaining a substantially constant flowrate while a tool is in the working channel of the endoscope. It is contemplated that in some cases, the flowrate may drop as the pressure dissipates.

The controller 30 may be configured to similarly control the motor speed of the inflow pump 60 in response to the intraluminal pressure (e.g., received from the pressure sensor 208 at the distal end region 206 of the endoscope 200). For example, if the intraluminal pressure remains below the maximum allowable pressure, the controller 30 may operate the motor of the inflow pump 60 at a constant RPM to maintain a constant flow of fluid to the working site. If the intraluminal pressure approaches the maximum allowable pressure, the controller 30 may slow, turn off, or reverse the motor of the inflow pump 60 to allow the pressure to dissipate or reduce.

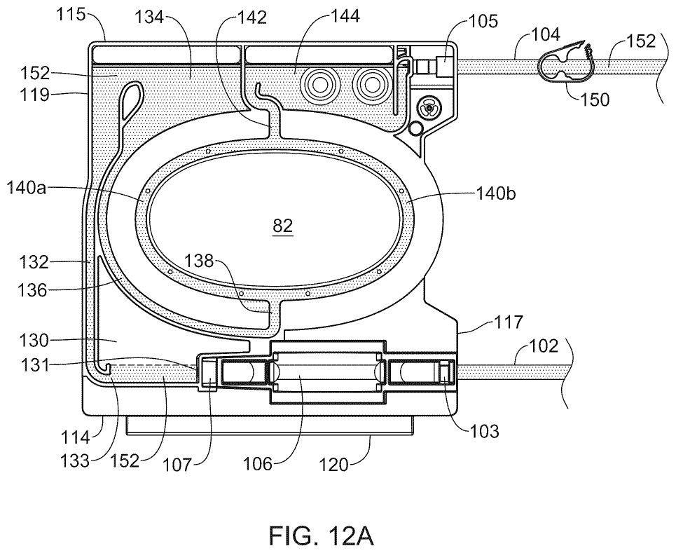

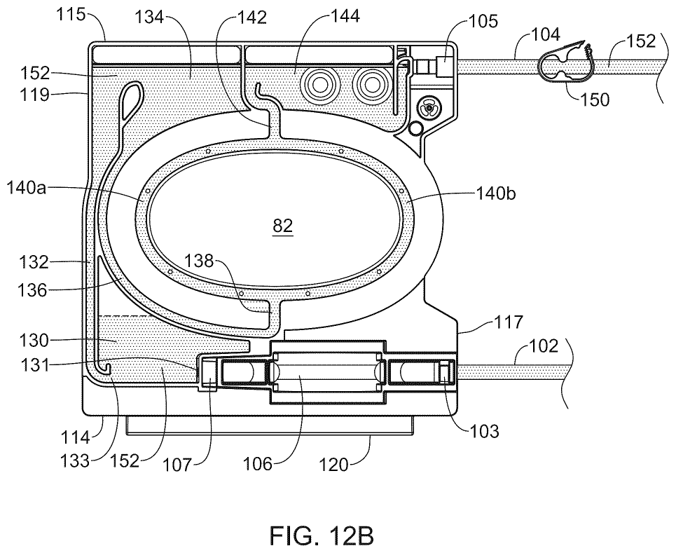

It is contemplated that at least some of the pump pressure may be stored in the form of compressed air in the fluid damping chamber 130 of the fluid cassette 110. FIG. 12A is a cross-sectional view of a portion of the fluid cassette of FIGS. 3 and 4 showing the internal fluid pathway with low inflow pump 60 pressure and FIG. 12B is a cross-sectional view of a portion of the fluid cassette of FIGS. 3 and 4 showing the internal fluid pathway with high inflow pump 60 pressure. As can be seen in FIGS. 12A and 12B, as the pressure increases, the amount of fluid 152 within the fluid damping chamber 130 increases. As the volume of the fluid 152 increases, the air in the fluid damping chamber 130 is compressed (e.g., pressure increases). When pressure is released or relieved from the fluid pathway, the volume of fluid 152 in the fluid damping chamber 130 decreases. Pressure release events may include, but are not limited to, releasing or opening one or more clamps 150 positioned along the outflow tubing 104, removing a tool from the working channel of the endoscope 200, or the like. For example, when a clamp 150 on the outflow tubing 104 is closed during flow compensation, the pressure of inflow pump 60 may continue to build until the pump pressure reaches the pressure setpoint plus the offset (in one example, setpoint plus 150 mmHg). When the clamp 152 on the outflow tubing 104 is unclamped or released, there may be a quick fluid discharge. If the endoscope 200 is inside the patient at that time, the fluid pushed out of the fluid cassette 110 is released into the patient. To reduce the likelihood of this occurring, a clamp on the outflow tubing 104 nearest to the scope may be removed. It is contemplated that reducing the pump pressure from an absolute value of 600 mmHg to 150 mmHg above the pressure setpoint may release less fluid if a clamp 150 is released with the fluid pathway under pressure. It is further contemplated that the compliance in the fluid pathway may be reduced to reduce the risk of a quick fluid discharge when a clamp 150 is released along the outflow tubing 104. Reducing the compliance in the fluid pathway may allow for an increased maximum allowed pump pressure and thus increased flow compensation.

In some embodiments, the controller 30 may be configured to improve the responsiveness of the flow compensation (e.g., adjustment of the pump speed to maintain a desired flowrate) using characterization and/or detection of tools. In one example, the controller 30 may be configured to characterize outflow resistance at the beginning of the procedure. This may be done by turning flow on (e.g., activating the inflow pump 60) and inserting the endoscope 200 to the desired treatment location. The controller 30 may then use the intraluminal pressure and pump speed to determine the outflow resistance. The outflow resistance may then be used to enhance flow compensation responsiveness and/or intraluminal pressure limiting. In another example, the controller 30 may determine a type of tool being inserted into the working lumen (e.g., automatically detect, determine based on a procedure type, or user selection at the touch screen interface 42). The controller 30 may then determine an expected flow reduction for the particular tool and use the expected flow reduction to enhance flow compensation. In some cases, the controller 30 may be preloaded with flow models for commonly used tools.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system, the system comprising:
a fluid management console, comprising:
a housing;
a controller housed within the housing;
an inflow pump disposed within the housing; and
a user input interface; and
a fluid cassette configured to be received within a receptacle of the housing of the fluid management console, the fluid cassette configured to provide a flow of fluid to a medical device;
wherein the controller of the fluid management console is configured to control the inflow pump to provide a flow of fluid at a predetermined flowrate.

2. The fluid management system of claim 1, wherein if a pump pressure reaches a predetermined pump pressure, the controller is configured to adjust a speed of a motor of the pump.

3. The fluid management system of claim 1, wherein if an intraluminal pressure reaches a predetermined intraluminal pressure, the controller is configured to adjust a speed of a motor of the pump.

4. The fluid management system of claim 1, wherein controlling the inflow pump comprises reducing a revolutions per minute (RPM) of a motor of the pump.

5. The fluid management system of claim 1, wherein controlling the inflow pump comprises stopping a motor of the pump.

6. The fluid management system of claim 1, wherein controlling the inflow pump comprises reversing a direction of a motor of the pump.

7. The fluid management system of claim 2, wherein the predetermined pump pressure is less than a maximum pump pressure.

8. The fluid management system of claim 7, wherein the maximum pump pressure is a setpoint pump pressure plus an offset.

9. The fluid management system of claim 8, wherein the offset is 150 mmHg.

10. The fluid management system of claim 3, wherein the predetermined intraluminal pressure is less than a maximum intraluminal pressure.

11. The fluid management system of claim 1, wherein the controller is configured to maintain the predetermined flowrate when a tool is inserted into a working channel of the medical device.

12. The fluid management system of claim 1, wherein the controller is configured to maintain the predetermined flowrate when an outflow resistance is increased above a baseline outflow resistance.

13. The fluid management system of claim 1, wherein the controller is configured to control the inflow pump to provide a flow of fluid at the predetermined flowrate by increasing and/or decreasing a pump pressure.

14. The fluid management system of claim 13, wherein when the pump pressure is above a setpoint pump pressure, a notification is displayed on the user input interface.

15. The fluid management system of claim 13, wherein the pump pressure is increased by a restriction in flow of a fluid.

16. A fluid management system, the system comprising:
a fluid management console, comprising:
a housing;
a controller housed within the housing;
an inflow pump disposed within the housing; and
a user input interface; and
a fluid cassette configured to be received within a receptacle of the housing of the fluid management console, the fluid cassette configured to provide a flow of fluid to a medical device;
wherein the controller of the fluid management console is configured to allow a pump pressure of the inflow pump to increase above a setpoint pump pressure in response to a flow restriction to provide a flow of fluid at a predetermined flowrate.

17. The fluid management system of claim 16, wherein if the pump pressure reaches a predetermined pressure greater than the setpoint pressure, the controller is configured to adjust a speed of a motor of the pump.

18. The fluid management system of claim 16, wherein if an intraluminal pressure reaches a predetermined intraluminal pressure greater than the setpoint pressure, the controller is configured to adjust a speed of a motor of the pump.

19. A fluid management system, the system comprising:
a fluid management console, comprising:
a housing;
a controller housed within the housing;
an inflow pump disposed within the housing and having a pump motor; and
a user input interface; and
a fluid cassette configured to be received within a receptacle of the housing of the fluid management console, the fluid cassette configured to provide a flow of fluid to a medical device;
wherein when a pump pressure is below a predetermined pump pressure and an intraluminal pressure is below a predetermined intraluminal pressure, the controller of the fluid management console is configured to operate the pump motor at a fixed revolutions per minute.

20. The fluid management system of claim 19, wherein if either the pump pressure increases above the predetermined pump pressure or the intraluminal pressure increases above the predetermined intraluminal pressure, the controller is configured to slow, stop, or reverse the pump motor.
